(19) United States
(12) Patent Application Publication
(10) Pub. No.: US 2004/0197789 A1
(43) Pub. Date: Oct. 7, 2004

(54) Detecting microoragnisms of the yersinia pestis/yersinia pseudotubercolosis species and/or differentiating between yersinia pestis and yersinia pseudotubercolosis

(76) Inventors: Simone Kohlaussen, Potsdam (DE); Kornelia Berghof, Berlin (DE); Reiner Grabowski, Weil (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/478,088

(22) PCT Filed: May 21, 2002
(86) PCT No.: PCT/EP02/05580

(30) Foreign Application Priority Data
May 18, 2001 (DE) 101 24 342.1

(51) Int. Cl. C12Q 1/68; C12P 19/34

(52) U.S. Cl. 435/6; 435/91.2

(57) ABSTRACT

Method and nucleic acids for the detection of microorganisms of the species Yersinia pestis/Yersinia pseudotuberculosis and/or differentiation between Yersinia pestis and Yersinia pseudotuberculosis in a sample.

Safe detection and/or differentiation methods for microorganisms of the species Yersinia pestis/Yersinia pseudotuberculosis are to be provided.

The method comprises several steps: (a) bringing the sample into contact with a combination of at least two first nucleic acids (primers) which hybridise with an area of their microbial genome or a part of it preserved in the species Yersinia pestis/Yersinia pseudotuberculosis; (b) the amplification of the microbial genome or part of it for production of at least one amplification fragment; (c) bringing the amplification fragment(s) obtained in step (b) into contact with at least one second nucleic acid (probe) which contains a specific partial sequence of the amplified genome or part of it for microorganisms of the species Yersinia pestis/Yersinia pseudotuberculosis, whereby the second nucleic acid (probe) hybridises specifically with at least one amplification fragment while forming at least one hybrid nucleic acid; (d) detection of the at least one hybrid nucleic acid.

The present invention relates to a method of detecting microorganisms of the Yersinia pestis/Yersinia pseudotuberculosis species and/or differentiation between Yersinia pestis and Yersinia pseudotuberculosis.

The plague caused by Yersinia pestis is today still a danger to public health Throughout the world, there are still natural reservoirs in many countries in Africa, America and Asia. In the period from 1990 to 1997, the number of registered cases of plague rose from 1257 to 5419 (WHO, 1999). The increase in international travel is increasing the risk of the disease spreading from endemic regions through infected travellers. A precise, rapid diagnosis of the plague would allow rapid intervention in the event of an outbreak of plague. Here, the knowledge of the strains currently circulating in every centre is essential if effective measures are to be taken (Leal et al., 1999, Rev. Inst Med. Trop. Sao Paulo 41, 339-342).

The genus Yersinia belongs to the family of the Enterobacteriaceae. Currently, this comprises 11 species, of which the species Yersinia pestis/Yersinia pseudotuberculosis and Yersinia enterocolitica are pathogenic in humans. Y. pestis and Y. pseudotuberculosis show a 90% DNA sequence homology, but both only show a 50% homology with Y. enterocolitica. Y. pestis and Y. pseudotuberculosis are therefore considered to be pathovars of a single species. In spite of the high sequential homology, however, there are fundamental differences between Y. pestis and Y. pseudotuberculosis, such as the method of transmission and the different lethality rates of the diseases caused and, as a result, a difference in the historical significance of the pathogens. However, the pathovars have not been reclassified as a subspecies (Achtman et al., 1999, Proc. Natl. Acad. Sci. USA 96, 14043-14048).

Yersinia pestis (syn. Pasteurella pestis), called after the French bacteriologist A. J. E. Yersin who first isolated the bacterium in 1894, is a non-flagellated, non-spore-forming, pleiomorphic, gram-negative bacterium. It is the agent which causes bubonic plague and can be transmitted to humans by fleas on infected rats and other rodents (field mice, susliks, marmots, squirrels). Earlier, it was found throughout the world, but today it only occurs in isolated enzootic and epizootic foci (mountain forests and savannah regions in America, Central, East and South Africa, Madagascar, Central and Southeast Asia). Yersinia pestis is able to survive for months in sputum, faeces, pus or dried in ectoparasites, which explains the spontaneous residual outbreaks (which also occur in apes, palm civets, camels and sheep). The three known biovars (by antiqua, by medievalis, by orientalis or oceanic) differ in their geographical distribution spectrum and reservoir. Completely pathogenic Y. pestis isolates are characterised by the presence of three different plasmids. The 9.5 kb plasmid pPla, which is specific to Y. pestis (also known as pPCP1 or pPst) carries a plasminogen activator/coagulase gene (pla gene) and a pesticin gene (Neubauer et al., 2000, J. Vet. Med., B47, 573-580). The second plague-specific plasmid pFra (or pMT1) is 110 kb in size. This codes for the F1 capsule antigen, amongst others. The third plasmid pYV (or pCD1 or pCad) is 70 kb large and occurs in all pathogenic isolates of the human pathogenic species Y. pestis/Y. pseudotuberculosis and Y. enterocolitica. The gene sequences which are already known here are saved in the GenBank Sequence Database at the National Centre for Biotechnology Information (NCBI) (pla-Gen (in the plasmid pPCP1): accession number AF053945-1; F1 antigen: accession number X61996; 16S ribosomal RNA: accession number L37604). Four clinical forms of plague are described (plague sepsis, bubonic, lung and abortive plague). Untreated, the lethality of bubonic plague is approx. 3040%. For this reason, even a suspicion of the disease is reason to begin antibiotic treatment immediately.

Yersinia pseudotuberculosis is a pathovar that is widespread amongst small rodents, cats and birds which is only of minor human medical significance. It is a pleiomorphic, peritrichal non-flagellated motile short rod. Human infections are always caused by diseased animals. Transmission is probably directly by smear infection or possibly indirectly via contaminated food. The agents are then ingested orally. Human pseudotuberculosis only occurs sporadically and very rarely. The generally benign course of the disease does not require any specific therapy.

The detection and identification of Yersinia pestis with conventional microbiological methods is firstly very time-consuming and in addition, because of the high pathogeneity of Y. pestis (L3), associated with risks for the laboratory personnel in the analytical laboratory. Secondly, the increased occurrence of resistances to particular antibiotics in human pathogenic agents increases the need for a treatment specifically adapted to the agent in question and thus also the need for a fast, clear identification of the agent. There is thus a considerable need for fast detection methods for human pathogenic agents such as Y. pestis.

A number of new methods have been developed in recent years for routine use in the detection of human pathogenic microorganisms. These include, for example, immunological methods based on the use of polyvalent or monoclonal antibodies, and methods in which nucleic acid probes are used for the detection of agent-specific nucleic acids by means of hybridisation. Further methods described are those methods that are based on a specific nucleic acid amplification, with or without subsequent confirmation reaction by nucleic acid hybridisation. Suitable methods for the amplification of nucleic acids are, for example, the polymerase chain reaction, PCR (U.S. Pat. Nos. 4,683,195, 4,683,202, 4965118), the ligase chain reaction (WO 89/09835), the self-sustained sequence replication (EP 329822), the transcription based amplification system (EP 310229), the Qβ RNA replicase system (U.S. Pat. No. 4,957,858) or an isothermic nucleic acid amplification. The nucleic acid based methods listed are so sensitive that, in contrast to conventional microbiological methods, the laborious enrichment of the microorganism to be detected from the sample to be tested is not necessary.

However, detection of the pathogenic agents (DNA) using PCR and subsequent display of the PCR products using gel electrophoresis alone are often not sufficient, since this does not provide a specific detection of the amplificates. An analysis using gel electrophoresis only shows the size and volume of the amplificate formed, but not the sequence (“identity”) of the amplification product.

For Yersinia pestis a number of specific DNA sequences are already known. In 1993, a first “nested” PCR was established by Campbell et al. (1993, J. Clin. Microbiol. 31, 758-759) from the plasminogen activator gene for the detection of Yersinia pestis, and a little later that year, Hinnebusch and Schwan (1993, J. Clin. Microbiol. 31, 1511-1514) also published a PCR based detection method for the same gene area. The authors amplified only one gene area, which can lead to falsely negative results if this gene area is missing or only partially present. Tsukano et al. (1996, Microbiol. Immunol. 40, 773-775) use a multiplex PCR system to detect Y. pestis. These detection systems have the disadvantage that they only use Yersinia plasmids as diagnostic target molecules. However, plasmids can easily be lost or be transferred to other bacteria, especially enterobacteria (Allen et al., 1987, Contr. Microbiol Immunol., 9, 332-341). The detection of plasmids alone therefore does not guarantee reliability in the identification of Yersinia pestis.

Another disadvantage of all the known methods is also that if nucleic acid sequences are used as the primer in the polymerase chain reaction, falsely negative results may occur. This has already been shown with other systems for the detection of pathogenic agents such as the bovine leukaemia virus (BLV-PCR) and feline infectious peritonitis virus (FIPV-RT-PCR) (Ballagi-Pordany et al, 1996, Mol. Cell Probes 10, 159-164). The detection of several different nucleic acid sequences, especially of “virulence markers” was also recorded by Leal et al (1999, Rev. inst Med. Trop. Sao Paulo 41, 339-342). However, because the detection system also uses plasmids, its specificity is not guaranteed. In addition, the sensitivity of this PCR system is not adequate to meet the requirements of industrial quality management.

Neubauer et al. (2000, J. Vet. Med. B. Infect. Dis. Vet Public Health 47, 573-580) also combined various “virulence markers”. In addition, a section of the 16S rDNA is detected as a “chromosomal marker”. The four target gene sections are detected here, not by multiplex PCR, but using separate PCR approaches. Testing the functionality of this Yersinia pestis PCR system (Neubauer et al.), however, shows that there are primers which only partly amplify (F1 antigen) the target sequence, or which do not amplify it at all (16 S). In addition, with these and the most recently mentioned detection systems for Y. pestis, no specificity control was implemented. Falsely positive or falsely negative results are therefore also possible here.

The object of the present invention is therefore to provide safe detection and/or differentiation methods for microorganisms of the species Yersinia pestis/Yersinia pseudotuberculosis.

This object is solved according to the invention by a method for the detection of microorganisms of the species Yersinia pestis/Yersinia pseudotuberculosis and/or differentiation of the pathovars Yersinia pestis and Yersinia pseudotuberculosis in a sample, whereby the method comprises the following steps:

(a) Bringing the sample into contact with a combination of at least two first nucleic acids (primers), which hybridise with an area of their microbial genome preserved in the species Yersinia pestis/Yersinia pseudotuberculosis or part thereof;

(b) Amplification of the microbial genome or a part of it to generate at least one amplification fragment;

(c) Bringing the amplification fragment(s) obtained in step (b) into contact with at least one second nucleic acid (probe) which comprises a specific partial sequence of the amplified genome or a part thereof for microorganisms of the pathovars Yersinia pestis and/or Yersinia pseudotuberculosis, whereby the second nucleic acid hybridises specifically with at least one amplification fragment while forming at least one hybrid nucleic acid;

(d) Detection of the at least one hybrid nucleic acid.

The method according to the invention can be carried out more safely and quickly than existing microbiological detection methods and allows the detection of microorganisms of the species Yersinia pestis/Y. pseudotuberculosis present in a sample and the differentiation of the pathovars covered by the species within a few hours. It also contains a specificity control which allows a safe identification of the reaction products and thus minimises the danger of falsely positive or falsely negative results. The method according to the invention allows the detection reaction to be automated and Yersinia pestis/Yersinia pseudotuberculosis to be quantified in the study material within just one working day.

In the following description, “identity” should be understood as the degree of relatedness between two or more nucleic acids, peptides or polypeptides which can be determined by the agreement between the sequences using known methods, e.g. computer-aided sequence comparisons (Basic local alignment search tool, S. F. Altschul et al., J. Mol. Biol. 215 (1990), 403-410). The percentage of the “identity” is determined from the percentage of identical areas in two or more sequences taking into account gaps or other sequence peculiarities. Preferred methods to determine the identity firstly create the greatest agreement between the studied sequences. Computer programs to determine the identity between two sequences include but are not limited to the GAG program package, including GAP (Devereux, J., et al., Nucleic Acids Research 12 (12): 287 (1984); Genetics Computer Group, University of Wisconsin, Madison, (WI)); BLASTP, BLASTN and FASTA (Altschul, S., et al., J. Mol. Biol. 215: 403-401) (1999)). The BLASTX program can be obtained from the National Centre for Biotechnology Information (NCBI) and from other sources (BLAST Manual, Altschul S., et al., NCB NLM NIH Bethseda MD 20894; Altschul, S., et al., Mol. Biol. 215: 403410 (1990)). The well-known Smith Waterman algorithm can also be used to determine identity.

Preferred parameters for the amino acid sequence comparison include the following:

| Algorithm: | Needleman and Wunsch, J. Mol. |
|---|---|
| | Biol. 48: 443-453 (1970); |
| Comparison matrix: | BLOSUM 62 from Henikoff and |
| | Henikoff, PNAS USA 89 (1992), |
| | 10915-10919; |
| GAP penalty: | 12; |
| GAP length penalty: | 4; |
| Threshold of similarity: | 0. |

The GAP program is also suitable for use with the above parameters. The above parameters are the error parameters (default parameters) for amino acid sequence comparisons, where gaps at the ends do not reduce the identity value. With very short sequences in comparison with the reference sequence, it may still be necessary to increase the expectation value to up to 100,000 and if necessary to reduce the word size to up to 2.

Further exemplary algorithms, gap opening penalties, gap extension penalties, comparative matrices including those given in the Program Manual, Wisconsin package, version 9, September 1997, can be used. The choice will depend on the comparison to be carried out and also on whether the comparison is carried out between sequence pairs, when GAP or Best Fit are preferred, or between a sequence and an extensive sequence database, when FASTA or BLAST are preferred.

An agreement of 70% determined with the above mentioned algorithm is described within the framework of this application as 70% identity. The same applies for higher identity levels.

The term “hybridisation” is understood as the double strand formation of two identical or similar nucleic acid fragments (DNA, RNA, PNA). Specific hybridisation is the term used if the hybridisation is carried out under strict conditions and gives a stable hybrid nucleic acid. In the terms of this invention, the feature “sequence which specifically hybridises with a sequence according to (i)” refers to a sequence which hybridises under strict conditions with the sequence according to (i). For example, the hybridisations can be carried out at 50-, or 55° C. with a hybridisation solution consisting of 2.5×SSC, 2× Denhardts solution, 10 mM Tris, 1 mM EDTA, pH 7.5. Suitable washing conditions are, for example, one-minute washes, repeated four times, in 0.1×SSC up to 1.0×SSC, 2× Denhardts, 10 mM Tris, 1 mM EDTA; pH 7.6 at 20-55° C.

The term “microbial genome” is understood as all the inherited information of a microorganism. It therefore includes the microbial chromosome and the existing episomal DNA.

A “nucleic acid” is a DNA, RNA or PNA which is obtained either through isolation from genomic DNA or from cDNA according to known standard methods (Sambrook et al., 1989) and purified or generated artificially using known methods (Sambrook et al., 1989) such as oligonucleotid synthesis or isolated as ribosomal RNA or mRNA from the organism or synthesised as PNA. A “PNA” is a peptide nucleic acid in which instead of the phosphoric acid backbone of the DNA, 2-aminoethylglycin compounds occur. According to the invention, in the nucleic acids, up to 20% of the nucleotides in 10 consecutive nucleotides, preferably however 1 nucleotide from a block of 10 consecutive nucleotides, may be replaced by nucleotides (e.g. inosin, etc.) which do not naturally occur in bacteria.

The nucleic acids may further contain modifications which allow the production of a signal that can be detected directly or indirectly. The expert is aware of the following modifications here:

(i) radioactive modifications, i.e. radioactive phosphorylation or radioactive marking with sulphur, hydrogen, carbon, nitrogen;

ii) colored groups (e.g. digoxygenin, etc.);

(iii) fluorescent groups (e.g. fluorescein, etc.);

(iv) chemoluminescent groups;

(v) groups for the immobilisation at a solid phase (e.g. biotin, streptag, antibodies, antigens, etc.); and/or

(vi) groups which allow an indirect or direct reaction with the help of antibodies, antigens, enzymes and/or substances with an affinity to enzymes or enzyme complexes;

or combinations of modifications according to two or more of the modifications listed under (i) to (vi). The term “modification” as used in this invention is understood to mean directly or indirectly detectable groups or groups for immobilisation at a solid phase which are attached to the nucleic acid. Metal atoms, radioactive, colored or fluorescent groups are directly detectable groups. Immunologically or enzymatically detectable groups are indirectly detectable groups, such as antigens and antibodies, haptenes or enzymes or parts of enzymes with an enzymatic effect. These indirect groups are detected in subsequent reactions. Preference is given to haptenes which are linked to an oligonucleotide and which are detected in a subsequent antibody reaction.

The term “primer” as used in this invention is understood to mean nucleic acids, which, in a polymerase chain reaction (PCR) for example, can hybridise specifically or unspecifically to a target sequence. In a special embodiment in the terms of this invention, a primer is modified or marked in such a way as described above for nucleic acids. Primers comprise at least 10 nucleotides, preferably 15-50 nucleotides, and particularly preferably 16-26 nucleotides.

A “probe” in the terms of this invention is understood to be a nucleic acid which hybridises specifically with a desired nucleic acid. Depending on the result required, probes are used which react either with the amplificates of all Yersinia pestis/Yersinia pseudotuberculosis pathovars to be detected, with a single pathovar or with the amplification control. A probe is therefore complementary either to a part sequence of the microbial genome of the species Yersinia pestis/Yersinia pseudotuberculosis, which is preserved in both pathovars, or to a sequence occurring in just one pathovar or to an amplification control sequence. In a particular embodiment under the terms of this invention, a probe is modified or marked as described above for nucleic acids. A probe is at least 10 nucleotides long, preferably 15-50 nucleotides, and particularly preferably 20-25 nucleotides.

Sequences are described as “preserved” if they are at least 70% identical. A preserved area is thus an area for which there is between the pathovars Yersinia pestis and Yersinia pseudotuberculosis 70% identity or more, preferably 80% identity or more, even more preferably 90% identity or more, particularly preferably 95% identity or more and most preferably 99% identity or more.

According to the invention, a sample to be tested is brought into contact with a combination of at least two first nucleic acids (primers). The sample may be blood, serum, plasma, lymph, liquid from “plague swellings”, saliva, sputum, faeces, pus or isolates from ectoparasites.

The at least two first nucleic acids are generally a forwards and a backwards primer which allow an amplification of the area delimited by them. Here, they hybridise with an area of a microbial nucleic acid from the sample which is preserved in the human pathogenic pathovars of the species Yersinia pestis/Yersinia pseudotuberculosis and which thus allows an amplification independently of the existing pathovar of the species Yersinia pestis/Yersinia pseudotuberculosis. The sequences of the pathovars complementary to the primer have at least a 70% identity in each case with the primer. In a further preferred embodiment of the present invention, primer sequences are used which hybridise with the genomic sequences from the pathovars Yersinia pestis/Yersinia pseudotuberculosis, whereby the sequences of the pathovars corresponding to the primer show at least 80% identity with the primer, preferably 90%, even more preferably 95% and particularly preferably 99%.

According to the invention, in a further step, the sample to be tested from the first step is amplified with a combination of at least two first nucleic acids (primers). In this step, at least one amplification fragment is produced. The amplification can be carried out according to the invention with any desired method, e.g. a PCR (U.S. Pat. No. 4,683,195, U.S. Pat. No. 4,683,202, U.S. Pat. No. 4,965,188), a ligase chain reaction, a self-sustained sequence replication (EP 329 822), a transcription based amplification (EP 310 229), a β-RNA Replicase System (U.S. Pat. No. 4,957,858) or an isothermic nucleic acid amplification.

According to the invention, the amplification fragments obtained in the previous step of the method are brought into contact with at least a second nucleic acid (probe). In this, the probe/probes hybridise, by forming a hybrid nucleic acid, specifically with at least one amplification fragment of a microbial nucleic acid which comprises a sequence of the microbial nucleic acid which occurs in the human pathogenic pathovars Yersinia pestis and/or Yersinia pseudotuberculosis. Hybridisation occurs through the pairing of the probes with areas of the microbial nucleic acid which show an at least partially complementary base sequence. By selecting a suitable probe sequence, it is then possible to determine whether the probe should recognise both pathovars of the species Yersinia pestis/Yersinia pseudotuberculosis or just one of these pathovars. In this way, it is possible either to detect human pathogen yersinia at all or to determine a single pathovar.

According to the invention, in a further step, the at least one hybrid nucleic acid produced in the previous step, consisting of an amplification fragment and a second nucleic acid is detected. The hybrid nucleic acids can be detected using various DNA detection methods, such as blot techniques, detection of radioactive isotopes, fluorescence detection methods, optical detection methods or other detection methods.

In a preferred embodiment of the present invention, the primer is selected from a preserved area of the genome containing the bacterial 16S rDNA gene. A further preferred embodiment uses a preserved area from one of the plasmids pPla (or pPCP1 or pPst), pFra (or pMT1) and/or pYV (or pCD1 or pCad) contained in Yersinia pestis. Here, sequences from the plasminogen activator gene (pPla) or the F1 antigen (pFra) are preferred. Particular preference is given to the area between Pos. 7359 and 7838 (pPla) or 4879 and 5133 (pFra). The sequences according to the invention according to SEQ ID NO. 1-9 are derived from the plasminogen activator gene, the F1 gene and the 16S rDNA gene and correspond to the following positions (cf. Table 1):

TABLE 1
Origin of the sequences of SEQ ID NO. 1-9

| SEQ ID NO. | Gene | Accession no. | Gene region | Length |
|---|---|---|---|---|
| 1 | Plasminogen | AF053945 | 7359-7378 | 20 |
| | activator gene | | | |
| 2 | Plasminogen | AF053945 | 7819-7838 | 20 |
| | activator gene | | | |
| 3 | (F1) capsular | X61996 | 5110-5133 | 24 |
| | antigen, caf1 | | | |
| 4 | (F1) capsular | X61996 | 4879-4900 | 22 |
| | antigen, caf1 | | | |
| 5 | 16S rDNA | L37604 | 989-1004 | 16 |
| 6 | 16S rDNA | L37604 | 985-1004 | 20 |
| 7 | 16S rDNA | L37604 | 1375-1400 | 26 |
| 8 | 16S rDNA | L37604 | 1375-1391 | 17 |
| 9 | 16S rDNA | L37604 | 1419-1398 | 22 |

SEQ ID NO. 1-9 correspond to the following sequences (cf. Table 2):

TABLE 2

Sequences of SEQ ID NO. 1-9

| | |
|---|---|
| SEQ ID NO. | Sequence |

| | |
|---|---|
| 1 | ATCTTACTTTCCGTGAGAAG |
| 2 | CTTGGATGTTGAGCTTCCTA |
| 3 | GGATTATTGGTTAGATACGGTTAC |
| 4 | GGTGATCCCATGTACTTAACAT |
| 5 | GGCAGAGATGCTAAAG |
| 6 | ATTTGGCAGAGATGCTAAAG |
| 7 | CTCCCATGGTGTGACGGGCGGTGTGT |
| 8 | TGTGACGGGCGGTGTGT |
| 9 | CTACTTCTTTTGCAACCCACTC |

In the sequences according to SEQ ID NO. 1-13, nucleotides are abbreviated as follows: G=guanosine, A=adenosine, T=thymidine, C=cytosine.

According to the invention, a combination of at least two first nucleic acids is used. The combination used of at least two first nucleic acids is chosen in such a way that they can be used as primers in an amplification reaction, i.e. a nucleic acid hybridises to a first preserved area of the first strand of the target DNA and the other nucleic acid to a second preserved area of the DNA strand complementary to the first strand, whereby the required target area of the DNA is enclosed. Both nucleic acids have a length of at least 10 nucleotides each, preferably 15-50 nucleotides, and even more preferably 15 to 30 nucleotides. In a further preferred embodiment of the invention, a combination of at least two pairs of first nucleic acids according to this invention are used, which therefore together lead to two amplified fragments. In a particular preferred embodiment, a first pair of nucleic acids hybridises with the bacterial 16S rDNA, whilst a second and if necessary a third or further primer pair hybridises with sequences from pPla, pFra or pYV.

Every first nucleic acid (primer) is selected from: (i) a nucleic acid that contains a nucleic acid sequence according to SEQ ID NO. 1-9 or an at least 10 and preferably 15 to 25 nucleotide long fragment of the same; (ii) a nucleic acid which hybridises specifically with a nucleic acid according to (i); (iii) a nucleic acid which is at least 70% identical with a nucleic acid according to (i) or (ii) or (iv) a nucleic acid which is complementary to a nucleic acid according to (i) to (iii).

In a preferred embodiment of the invention, the method for the amplification contains a polymerase chain reaction (PCR). The polymerase chain reaction can also be carried out as a multiplex PCR. The “multiplex PCR” is a detection of various target molecules in a single polymerase chain reaction, in which a mixture of primers is used. In a preferred embodiment of the invention, in a multiplex PCR, four first nucleic acids are used as primers. An online or real-time PCR is also possible. With the online or real-time PCR, the result of the amplification is shown at the same time as it occurs. In an ideal situation, as with the LightCycler, for example, the amplification can also be detected in real time. A major advantage of real-time PCR is thus the speed with which the result is obtained. Because secondary work stages, such as the detection of the amplification products using ELISA or gel electrophoresis, are not necessary, the amount of work is considerably reduced. Finally, real-time PCR offers the advantage that it allows a “real” quantification of the DNA present in the sample and thus the number of microorganisms. If, instead of DNA, RNA is detected as the target molecule in an RT PCR, a distinction can be made between living and dead Yersinia pestis bacteria. The online PCR can also be carried out with one or several of the primers (SEQ ID NO. 1-9). In addition, the presence of one or more probes, e.g. from SEQ ID NO. 10-13, in the PCR reaction mixture is decisive for the function of the online PCR. The online PCR itself can be carried out in various ways. Basically, the only difference from other PCR methods is the occurrence of the fluorescence signal. The 5′ nuclease assay with TaqMan probes or Molecular Beacons, for example, ca be used. Another possibility is to use LightCycler probes. In the latter case, two fluorescence marked probes are used and the signal is created by an energy transfer from the dye of the one probe to the dye of a neighbouring probe. For this reason, alongside the probes of the SEQ ID NO. 10-13, a “neighbouring probe” is selected from the known PCR target sequences of Yersinia pestis.

In a preferred embodiment of the present invention, the detection of human pathogen yersinia is carried out using probe sequences which hybridise with genomic sequences from the pathovars Yersinia pestis and Yersinia pseudotuberculosis, whereby the sequences of the pathovars complementary to the probe are at least 80% identical to each other. Preferably, probe sequences are used which hybridise with genomic sequences from the pathovars Yersinia pestis and Yersinia pseudotuberculosis, whereby the sequences of the pathovars complementary to the probe are at least 90% identical to each other, preferably 95% and particularly preferably 99%. In a preferred embodiment of the present invention, the probe is selected from a preserved area of the genome which contains the bacterial 16S rDNA genes. A further preferred embodiment uses a preserved area from one of the plasmids pPla (or pPCP1 or pPst), pFra (or pMT1) and/or pYV (or pCD1 or pCad) contained in Yersinia pestis. Here, sequences from the plasminogen activator gene (pPla) or the F1 antigen (pFra) are preferred.

For the detection of a single pathovar, on the other hand, probes are preferred which have an identity of 80% or more, preferably 90%, 95%, 99% or more with only one of the pathovars, whilst the identity with the other pathovar must be correspondingly smaller in order to be able to observe a selective hybridisation. Preferably, the difference in degree of identity is at least 5%, but more preferably 10% or 15% and particularly preferably more than 20 or 25%.

Areas particularly preferred for probes are the areas between pos. 7583 and 7602 (pPla) and/or 5008 and 5027 (pFra). The sequences preferred according to the inventions according to SEQ ID NO. 10 to 12 are derived from the plasminogen activator gene, the F1 gene and the 16S rDNA gene and correspond to the following positions (cf. Table 3):

TABLE 3
Origin of the sequences of SEQ ID NO. 10-12

| SEQ ID NO. | Gene | Accession no. | Gene region | Length |
|---|---|---|---|---|
| 10 | 16S rDNA | L37604 | 1178-1198 | 21 |
| 11 | (F1) capsular | X61996 | 5008-5027 | 20 |
| | antigen, caf1 | | | |
| 12 | Plasminogen | AF053945 | 7583-7602 | 20 |
| | activator gene | | | |

SEQ ID NO. 10-12 correspond to the following sequences (cf. Table 4):

TABLE 4

Sequences of SEQ ID NO. 10-12

| | |
|---|---|
| SEQ ID NO. | Sequence |

| | |
|---|---|
| 10 | AGTCATCATGGCCCTTACGAG |
| 11 | GATGACGTCGTCTTGGCTAC |
| 12 | GGTCTGCAATATCGCTTCTG |

In the sequences according to SEQ ID NO. 1-12, nucleotides are abbreviated as follows: G=guanosine, A=adenosine, T=thymidine, C=cytosine.

Probes have a length of at least 10 nucleotides, preferably 15-50 nucleotides, and even more preferably 15 to 30 nucleotides. Preferred probes are chosen from:

(v) a nucleic acid that contains a nucleic acid sequence according to SEQ ID NO. 10-12 or an at least 10 and preferably 15 to 20 nucleotide long fragment of the same;

(vi) a nucleic acid which hybridises specifically with a nucleic acid according to (v);

(vii) a nucleic acid which is at least 80% identical with a nucleic acid according to (v) or (vi); or

(viii) a nucleic acid which is complementary to a nucleic acid according to (v) to (vii).

Probes may also contain modifications which allow the production of a signal that can be detected directly or indirectly.

The probes of SEQ ID NO. 10-12 have a different specificity for the pathovars Yersinia pestis and Yersinia pseudotuberculosis (cf. Table 6). Thus the probes of SEQ ID NO. 11-12 can be used to specifically detect the pathovar Yersinia pestis, whilst on the other hand, the probe of SEQ ID NO. 10 can be used to detect the pathovars Yersinia pestis and Yersinia pseudotuberculosis. This means that differentiation between the pathovars Yersinia pestis and Yersinia pseudotuberculosis is possible.

To avoid falsely negative results, in a preferred embodiment of the invention, an amplification control nucleic acid is added during amplification. Accordingly, not only the microbial nucleic acid or a part thereof is amplified, but also the amplification control nucleic acid, which means that at least one amplification fragment in each case is created. The amplification control nucleic acid, e.g. a DNA fragment, is here an “internal standard molecule” which serves as an indicator for the effectiveness of the reaction (Ballagi-Pordany, Belak, 1996, Mol. Cell. Probes 10, 159-164). It is added in a defined quantity to the amplification reaction and amplified in parallel. The amplification control nucleic acid is preferably single or double strand and may be of unlimited length. Amplification control nucleic acids with a length of up to a thousand nucleotides have proved successful. In a preferred embodiment of this invention, the amplification control nucleic acid contains a sequence that is identical or complementary to the sequence of SEQ ID NO. 13 or a part of it.

In a further preferred embodiment of this invention, the amplification takes place as a competitive PCR. In the competitive PCR, the target DNA and the amplification control DNA are amplified with the same primer pair. In a particularly preferred embodiment of the invention, the 3′ and 5′ areas of the amplification control DNA are formed in such a way that they can be amplified with a primer pair selected for the amplification of the target DNA. In this case, one talks of a competitive amplification control, since the target DNA and the control DNA are amplified with the same primer pair. In a preferred embodiment of this invention, the amplification control nucleic acid is amplified using at least two of the primers of SEQ ID NO. 5-9.

In a special embodiment of the method according to the invention, with the use of an amplification control, the amplified amplification control nucleic acid is brought into contact with a probe which hybridises specifically with at least one amplification fragment of the amplification control nucleic acid. The probe is selected from:

(ix) a nucleic acid that contains a nucleic acid sequence according to SEQ ID NO. 13 or an at least 10 and preferably 15 to 19 nucleotide long fragment of the same;

(x) a nucleic acid which hybridises specifically with a nucleic acid according to (ix);

(xi) a nucleic acid which is at least 80% identical with a nucleic acid according to (ix) or (x); or

(xii) a nucleic acid which is complementary to a nucleic acid according to (ix) to (xi).

A probe according to SEQ ID NO 13 may also contain modifications which allow the production of a signal that can be detected directly or indirectly. The probe according to SEQ ID No. 13 can be used separately or in combination with one or more probes according to SEQ ID NO. 10-12.

| | |
|---|---|
| SEQ ID NO. 13 | GACTACGGAATTCCGCTGTC |

According to the invention, at least one hybrid nucleic acid is detected consisting of an amplification fragment and a second nucleic acid incorporated in the previous step. The hybrid nucleic acids can be detected using various DNA detection methods, such as blot techniques, fluorescence detection methods, optical detection methods or other detection methods. In a preferred embodiment, the hybrid nucleic acids are detected using ELISA techniques. In a further preferred embodiment, the hybrid nucleic acid is detected using the Southern Blot method.

Multiplication of the microbial nucleic acid or a part of it and subsequent detection of these molecules can be carried out, for example, by means of hybridisation with marked specific probes. In a multiplex PCR, nucleic acids can be used which allow an amplification product to be obtained from several or even all the relevant strains, subspecies or species and various target molecules. The specificity of the detection is achieved by the subsequent hybridisation reaction with specific probes. In this way, Yersinia pestis and Y. pseudotuberculosis can be detected in the presence of an amplification control simultaneously in a simple combination of amplification and detection reaction. This type of amplification and detection allows the detection reaction to be automated so that a higher sample throughput is possible. For example, a PCR ELISA detection method can be used in which the corresponding probes are bound into various cavities of a microtitre plate in which the hybridisation and detection of the marked amplificates is then carried out. Detection can also be carried out by the use of a microarray on which several probes are immobilised, which means that the detection reaction can be carried out quickly and without major effort. The nucleic acids according to the invention can thus be used for the detection and/or identification and/or characterisation of Yersinia pestis or Y. pseudotuberculosis. The primers and/or probes described here can be used additionally in the detection of the bacteria described in various samples such as in environmental or clinical samples, etc.

The invention also contains a kit for the detection of microorganisms of the species Yersinia pestis/Yersinia pseudotuberculosis and for differentiation between Yersinia pestis and Yersinia pseudotuberculosis, containing two or more nucleic acids. The invention also includes in a special embodiment a kit for the detection of microorganisms of the species Yersinia pestis/Yersinia pseudotuberculosis and for differentiation between Yersinia pestis and Yersinia pseudotuberculosis containing two or more nucleic acids and an amplification control for the amplification reaction.

The following figures and examples elucidate the invention:

### DESCRIPTION OF FIGURES

FIG. 1: Sensitivity of the multiplex PCR system

FIG. 1 shows an inverted representation of the amplificates of genomic DNA

(Y. pestis) after amplification and separation by gel electrophoresis;

Kev: (traces from left to right):

M: Marker, trace 1 to 7: Y. pestis, 1 ng to 1 fg initially used genomic DNA, trace 8: negative control;

Pla: amplificate plasminogen activator gene;

16S or ST: amplificate 16 S or control DNA;

F1: amplificate F1 antigen.

### EXAMPLES

### Example 1

### Detection of Human Pathogenic Bacteria Using the Polymerase Chain Reaction

I. Amplification

Genomic DNA was isolated from killed pure cultures of the bacteria listed in Table 5 using known standard methods. Dilutions (concentration in the area of approx. 1 ng-1 pg) of these preparations were then added to a multiplex PCR with the following composition:

Primer 1 = SEQ ID NO. 1
Primer 2 = SEQ ID NO. 2
Primer 3 = SEQ ID NO. 3
Primer 4 = SEQ ID NO. 4
Primer 5 = SEQ ID NO. 6
Primer 6 = SEQ ID NO. 9

| | Volume | Concentration |
|---|---|---|
| Components | [μ] | in preparation |
| H₂O | to 25 | — |
| PCR buffer | 2.5 | 1 × conc. |
| MgCl₂ | 2.0 | 0.5-5.0 mM |
| dTTP mix | 0.5 | 10-100 μM each |
| Primers: | | |
| SEQ ID NO. 1 | 0.5 each | 0.2-1.0 μM |
| SEQ ID NO. 2* | | |
| SEQ ID NO. 3* | | |
| SEQ ID NO. 4 | | |
| SEQ ID NO. 6 | | |
| SEQ ID NO. 9* | | |
| Taq polymerase | 0.2 | 1-5 U/μl |
| DNA extract/eluate | 1.0 | var. |
| from sample | | |
| Control DNA | 1.0 | var. |
| Σ | 25 μl | |

*= primers are 5′ digoxigenin marked

The PCR was carried out under the following conditions in the Perkin Elmer Thermocycler, model 9700:

| Step | Time | Temperature |
|---|---|---|
| Initial denaturation | 5 min | 95° C. |

“touch down”: 5 × (−2° C.)

| | | |
|---|---|---|
| Denaturation | 20-60 sec | 94° C. |
| Accumulation | 20-60 sec | 68° C.-58° C. |
| Synthesis | 30-90 sec | 72° C. |

30-35 x

| | | |
|---|---|---|
| Denaturation | 20-60 sec | 94° C. |
| Accumulation | 20-60 sec | 58° C. |
| Synthesis | 30-90 sec | 72° C. |
| Final synthesis step | 5 min | 72° C. |
| Final temperature | | 4° C.-10° C. |

Primer 3 (SEQ ID NO. 3) and primer 4 (SEQ ID NO. 4) were determined through the sequence comparison of known F1 antigen sequences (GenBank Sequence Database of the NCBI). They hybridise to highly preserved sequence sections in the F1 antigen gene area. Primer 5 (SEQ ID NO. 6) and Primer 6 (SEQ ID NO. 7 or 9) were determined through the sequence comparison of known 16S rDNA sequences (GenBank Sequence Database of the NCBI) and own sequence data.

II. Detection in PCR ELISA

Detection is carried out in the PCR ELISA. For this, for each probe used, 5 μl amplificate is diluted with 5 μl denaturation buffer (pH 14; 100-200 mM NaOH, 10-25 mM EDTA) and incubated at room temperature for 10 minutes. For each sample, 1.5 pmol of the relevant biotinylated probe (SEQ ID NO. 10-13) is pipetted into 100 μl hybridisation buffer (pH 7.5; 2-5×SSC, 1-5× Denhardts solution, 5-10 mM Tris, 0.1-1.5 mM EDTA). After denaturation, for each cavity of the microtitre plate, 10 μl of the denaturation mixture and 100 μl of the relevant probe hybridisation buffer mixture is transferred to the cavities of a microtitre plate coated with streptavidin. This is followed by a 30 minute incubation at hybridisation temperature (45-55° C.). Once the hybridisation has finished, the hybridisation mixture is removed and washed 4× with 200 μl washing buffer 1 (WB1: pH 7.6; 0.1-1×SSC, 1-5× Denhardts, 5-10 mM Tris, 0.1-1.5 EDTA) for 1 to 4 minutes each time at hybridisation temperature. After this, 100 μl of a solution, diluted according to the manufacturer's instructions, of an anti-digoxigenin antibody conjugated with a horseradish peroxidase is added (Boehringer Mannheim). The conjugate is diluted in washing buffer 2 (WB2: pH 7.5; 50-100 mM Tris, 100-200 mM NaCl, 0.01-0.1% Tween 20, 0.1-1.0% blocking reagent, 50-120 μg/ml herring sperm). This is followed by antibody incubation at 37° C. for 30 minutes. After this, washing is carried out (at room temperature) 4× with 200 μl washing buffer 2. After the washing, 100 μl POD substrate (Boehringer Mannheim) is added and incubated for 15 minutes at room temperature. The colour reaction is then stopped with 100 μl 0.3-0.7 M H₂SO₄ and measured at 450 nm against 650 nm in the ELISA reader.

III. Evaluation

According to the detection protocol described above, detection was carried out for all the bacteria studied using the corresponding specific probes. SEQ ID NO. 10-13 were used as specific probes for Yersinia pestis and Y. pseudotuberculosis.

If the measured extinction was greater than 0.50, the result was assessed as positive. The results of the PCR ELISA are shown in Table 6.

TABLE 5
Microorganisms used

| | Strain no./ | |
|---|---|---|
| Species | biovar | Abbreviation |
| Yersinia aldovae | ATCC 35236 | |
| Yersinia enterocolitica | DSM 4780 | |
| Yersinia frederiksenii | ATCC 33641 | |
| Yersinia intermedia | ATCC 29909 | |
| Yersinia kristensenii | ATCC 33638 | |
| Yersinia mollaretii | ATCC 43969 | |
| Yersinia pseudotuberculosis | DSM 8992 | |
| Yersinia pseudotuberculosis | NC 1102 | |
| Yersinia pseudotuberculosis | NC 824 | |
| Yersinia pseudotuberculosis | NC 1779 | |
| Yersinia pseudotuberculosis | NC 9507 | |
| Yersinia pseudotuberculosis | NC 10277 | |
| Yersinia pseudotuberculosis | NC 10278 | |
| Yersinia pseudotuberculosis | NC 8580 | |
| Yersinia pseudotuberculosis | NC 8579 | |
| Yersinia pestis | ATCC 19428 | 172 |
| Yersinia pestis | biotype “O” | EV76 |
| Yersinia pestis | biotype “O” | 6/69 |
| Yersinia pestis | biotype “O” | A1122 |
| Yersinia pestis | biotype “O” | M23 |
| Yersinia pestis | biotype “O” | G32 |
| Yersinia pestis | biotype “A” | Yokuhama |
| Yersinia pestis | biotype “A” | Kuma |
| Yersinia pestis | biotype “M” | KIM |
| Yersinia rohdei | ATCC 43380 | |
| Yersinia ruckeri | ATCC 29473 | |

TABLE 6
Results of the PCR ELISA (OD 450 nm)

| | | SEQ | | | SEQ |
|---|---|---|---|---|---|
| | | ID | | | ID |
| | | NO. | SEQ ID | SEQ ID | NO. |
| | | 10 | NO. 11 | NO. 12 | 13 |
| Strain | Probe | 16S | F1 | pla | ST |
| Yersinia aldovae | ATCC 35236 | 0.265 | 0.028 | 0.025 | 3.945 |
| Yersinia enterocolitica | DSM 4780 | 0.058 | 0.033 | 0.023 | 3.809 |
| Yersinia frederiksenii | ATCC 33641 | 0.293 | 0.025 | 0.025 | 3.630 |
| Yersinia intermedia | ATCC 29909 | 0.069 | 0.018 | 0.021 | 3.495 |
| Yersinia kristensenii | ATCC 33638 | 0.142 | 0.031 | 0.021 | 4.000 |
| Yersinia mollaretii | ATCC 43969 | 0.105 | 0.023 | 0.023 | 3.641 |
| Yersinia | DSM 8992 | 4.000 | 0.023 | 0.030 | 0.081 |
| pseudotuberculosis | | | | | |
| Yersinia | NC 1102 | 3.895 | 0.039 | 0.040 | 0.073 |
| pseudotuberculosis | | | | | |
| Yersinia | NC 824 | 4.000 | 0.039 | 0.036 | 0.069 |
| pseudotuberculosis | | | | | |
| Yersinia | NC 1779 | 3.841 | 0.042 | 0.036 | 0.161 |
| pseudotuberculosis | | | | | |
| Yersinia | NC 9507 | 4.000 | 0.040 | 0.038 | 0.050 |
| pseudotuberculosis | | | | | |
| Yersinia | NC 10277 | 3,859 | 0.040 | 0.038 | 0.041 |
| pseudotuberculosis | | | | | |
| Yersinia | NC 10278 | 4.000 | 0.041 | 0.038 | 0.040 |
| pseudotuberculosis | | | | | |
| Yersinia | NC 8580 | 4.000 | 0.037 | 0.037 | 0.132 |
| pseudotuberculosis | | | | | |
| Yersinia | NC 8579 | 3.934 | 0.038 | 0.033 | 0.071 |
| pseudotuberculosis | | | | | |
| Yersinia pestis | ATCC 19428 | 4.000 | 1.168 | 2.804 | 4.000 |
| Yersinia pestis | EV76 | 4.000 | 4.000 | 4.000 | 0.166 |
| Yersinia pestis | 6/69 | 4.000 | 4.000 | 4.000 | 0.065 |
| Yersinia pestis | A1122 | 4.000 | 4.000 | 4.000 | 0.061 |
| Yersinia pestis | M23 | 3.890 | 4.000 | 3.916 | 0.063 |
| Yersinia pestis | G32 | 4.000 | 4.000 | 1.970 | 0.056 |
| Yersinia pestis | Yokuhama | 4.000 | 4.000 | 4.000 | 0.045 |
| Yersinia pestis | Kuma | 4.000 | 4.000 | 4.000 | 0.086 |
| Yersinia pestis | KIM | 4.000 | 4.000 | 4.000 | 0.075 |
| Yersinia rohdei | ATCC 43380 | 0.120 | 0.019 | 0.020 | 3.810 |
| Yersinia ruckeri | ATCC 29473 | 0.154 | 0.020 | 0.020 | 3.743 |

The results in Table 6 show that the examined strains of the species Yersinia pestis and Y. pseudotuberculosis were identified.

### Example 2

### Sensitivity of the Detection of Human Pathogenic Bacteria with the Polymerase Chain Reaction

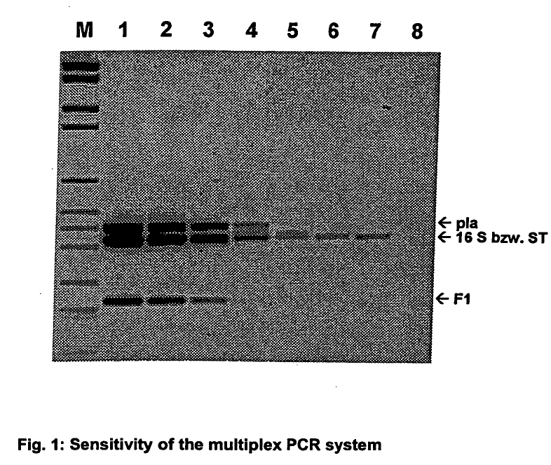

Amplication and detection in PCR ELISA and evaluation were carried out as described in Example 1. As sample material, in contrast to Example 1, the genomic DNA isolated from the strain Y. pestis ATCC 19428 was introduced in decadic dilutions into a multiplex PCR. The results are shown in FIG. 1 and Table 7.

TABLE 7
Results of the PCR ELISA (OD 450 nm)

| | SEQ ID | SEQ ID | SEQ ID | SEQ ID |
|---|---|---|---|---|
| Probe | NO. 10 | NO. 11 | NO. 12 | NO. 13 |
| DNA | pla | F1 | 16S | ST |
| 0 | 0.051 | 0.026 | 0.028 | 3.566 |
| 1 | 0.044 | 0.026 | 0.131 | 3.118 |
| 10 | 0.305 | 0.270 | 0.456 | 3.179 |
| 100 | 2.271 | 1.131 | 2.998 | 2.612 |
| 1000 | 4.000 | 3.537 | 4.000 | 2.694 |
| 10000 | 4.000 | 3.940 | 4.000 | 1.201 |
| 100000 | 4.000 | 4.000 | 4.000 | 0.254 |
| 1000000 | 4.000 | 4.000 | 4.000 | 0.057 |

1. Method for the detection of microorganisms of the species Yersinia pestis/Yersinia pseudotuberculosis and/or differentiation between the pathovars Yersinia pestis and Yersinia pseudotuberculosis in a sample, whereby the method comprises the following steps:
(a) bringing the sample into contact with a combination of at least two first nucleic acids (primers) which hybridize with an area of their microbial genome or part of it preserved in the species Yersinia pestis/Yersinia pseudotuberculosis;
(b) amplifying the microbial genome or a part of it to produce at least one amplification fragment;
(c) bringing the amplification fragment(s) obtained in step (b) into contact with at least a second nucleic acid (probe), which contains a specific partial sequence of the amplified genome or a part of it for microorganisms of the pathovars Yersinia pestis and/or Yersinia pseudotuberculosis, whereby the second nucleic acid (probe) specifically hybridizes with at least one amplification fragment whilst forming at least one hybrid nucleic acid; and
(d) detecting of at least one hybrid nucleic acid.

2. The method according to claim 1, characterized in that the first nucleic acid(s) (primer(s)) hybridize(s) with a preserved area of the plasmids pPla, pFra or pYV from Yersinia pestis/Yersinia pseudotuberculosis or a part thereof.

3. The method according to claim 1, characterized in that the first nucleic acid(s) (primer(s)) hybridize(s) with a preserved area of a plasminogen activator gene and/or an F1 gene from Yersinia pestis/Yersinia pseudotuberculosis.

4. The method according to claim 1, characterized in that the first nucleic acid(s) (primer(s)) hybridize(s) with a preserved area of a bacterial 16S rDNA gene or a part thereof.

5. The method according to claim 1, characterized in that the second nucleic acid(s) (probe) hybridise(s) with a preserved area of the plasmids pPla, pFra or pYV from Yersinia pestis/Yersinia pseudotuberculosis or a part thereof.

6. The method according to claim 1, characterized in that the second nucleic acid(s) (probe) hybridise(s) with a preserved area of a plasminogen activator gene and/or an F1 gene from Yersinia pestis/Yersinia pseudotuberculosis.

7. The method according to claim 1, characterized in that the second nucleic acid(s) (probes) hybridize(s) with a preserved area from the bacterial 16S rDNA gene or a part thereof.

8. The method according to claim 1, characterized in that one or more nucleic acids selected from the following group are used as the first nucleic acid(s) (primer(s)):
(i) a nucleic acid that contains a nucleic acid sequence according to SEQ ID NO. 1-9 or an at least 10 or 15 to 25 nucleotide long fragment of the same;
(ii) a nucleic acid which hybridizes specifically with a nucleic acid according to (i);
(iii) a nucleic acid which is at least 70% identical with a nucleic acid according to (i) or (ii); and
(iv) a nucleic acid which is complementary to a nucleic acid according to (i) to (iii).

9. The method according to claim 1, characterized in that one or more nucleic acids selected from the following group are used as the second nucleic acid (probe):
(v) a nucleic acid that contains a nucleic acid sequence according to SEQ ID NO. 10-12 or an at least 10 or 15 to 20 nucleotide long fragment of the same;
(vi) a nucleic acid which hybridises specifically with a nucleic acid according to (v);
(vii) a nucleic acid which is at least 80% identical with a nucleic acid according to (v) or (vi); and
(viii) a nucleic acid which is complementary to a nucleic acid according to (v) to (vii).

10. The method according to claim 1, characterized in that the nucleic acid is a DNA, RNA or PNA.

11. The method according to claim 1, characterized in that up to 20% of the nucleotides, but at least 1 nucleotide in 10 consecutive nucleotides, is replaced by nucleotides that do not occur naturally in bacteria.

12. The method according to claim 1, characterized in that a modification is introduced into the nucleic acid for production of a signal that can be detected directly or indirectly, whereby the modification may consist of (i) a radioactive marking, (ii) colored groups, (iii) fluorescent groups, (iv) chemoluminescent groups, (v) groups for immobilization at a solid phase and/or (vi) groups which permit an indirect or direct detection reaction using antibodies, antigens, enzymes and/or substances with an affinity to enzymes or enzyme complexes, or a combination of modifications according to two or more of the modifications listed under (i) to (vi).

13. The method according to claim 1, characterized in that the amplification includes a polymerase chain reaction (PCR).

14. The method according to claim 13, characterized in that the PCR comprises a multiplex PCR and is carried out with at least four first nucleic acids.

15. The method according to claim 13, characterized in that the PCR comprises an online or real-time PCR.

16. The method according to claim 1, characterized in that the amplification comprises a ligase chain reaction, an isothermic nucleic acid amplification or a Qβ replication.

17. The method according to claim 1, characterized in that an amplification control is carried out for the amplification.

18. The method according to claim 17, characterized in that the amplification control contains a DNA sequence of maximum 1,000 nucleotides.

19. The method according to claim 18, characterized in that the DNA sequence of the amplification control is selected from the following group:
(ix) a nucleic acid that contains a nucleic acid sequence according to SEQ ID NO. 13 or an at least 10 or 15 to 19 nucleotide long fragment of the same;
(x) a nucleic acid which hybridizes specifically with a nucleic acid according to (ix);
(xi) a nucleic acid which is at least 80% identical with a nucleic acid according to (ix) or (x); and
(xii) a nucleic acid which is complementary to a nucleic acid according to (ix) to (xii).

20. A nucleic acid for the detection of microorganisms of the species Yersinia pestis/Yersinia pseudotuberculosis and/or differentiation between Yersinia pestis and Yersinia pseudotuberculosis, chosen from:
(i) a nucleic acid that contains a nucleic acid sequence according to SEQ ID NO. 1-9 or an at least 10 or 15 to 25 nucleotide long fragment of the same;
(ii) a nucleic acid which hybridizes specifically with a nucleic acid according to (i);
(iii) a nucleic acid which is at least 70% identical with a nucleic acid according to (i) or (ii);
(iv) a nucleic acid which is complementary to a nucleic acid according to (i) to (iii);
(v) a nucleic acid that contains a nucleic acid sequence according to SEQ ID NO. 10-12 or an at least 10 or 15 to 20 nucleotide long fragment of the same;
(vi) a nucleic acid which hybridizes specifically with a nucleic acid according to (v);
(vii) a nucleic acid which is at least 80% identical with a nucleic acid according to (v) or (vi); or
(viii) a nucleic acid which is complementary to a nucleic acid according to (v) to (vii).

21. A nucleic acid for controlling an amplification, selected from:
(ix) a nucleic acid that contains a nucleic acid sequence according to SEQ ID NO. 13 or an at least 10 and preferably 15 to 19 nucleotide long fragment of the same;
(x) a nucleic acid which hybridizes specifically with a nucleic acid according to (ix);
(xi) a nucleic acid which is at least 80% identical with a nucleic acid according to (ix) or (x); or
(xii) a nucleic acid which is complementary to a nucleic acid according to (ix) to (xi).

22. The nucleic acid according to claim 20, characterized in that it is a DNA, RNA or PNA.

23. The nucleic acid according to claim 20, characterized in that the nucleic acid is modified by the fact that up to 20% of the nucleotides, but at least 1 nucleotide in 10 consecutive nucleotides, are replaced by nucleotides that do not occur naturally in bacteria.

24. The nucleic acid according to claim 20, characterized in that a modification is introduced into the nucleic acid for production of a signal that can be detected directly or indirectly, whereby the modification may consist of (i) a radioactive marking, (ii) colored groups, (iii) fluorescent groups, (iv) chemoluminescent groups, (v) groups for immobilization at a solid phase and/or (vi) groups which permit an indirect or direct detection reaction using antibodies, antigens, enzymes and/or substances with an affinity to enzymes or enzyme complexes, or a combination of modifications according to two or more of the modifications listed under (i) to (vi).

25. (cancelled)

26. (cancelled)

27. Kit for the detection of microorganisms of the species Yersinia pestis/Yersinia pseudotuberculosis and for differentiation between Yersinia pestis and Yersinia pseudotuberculosis containing two or more nucleic acids according to claim 20.

28. The kit according to claim 27 containing in addition an amplification control for the amplification reaction.

29. The nucleic acid according to claim 21, characterized in that it is a DNA, RNA or PNA.

30. The nucleic acid according to claim 21, characterized in that the nucleic acid is modified by the fact that up to 20% of the nucleotides, but at least 1 nucleotide in 10 consecutive nucleotides, are replaced by nucleotides that do not occur naturally in bacteria.

31. The nucleic acid according to claim 21, characterized in that a modification is introduced into the nucleic acid for production of a signal that can be detected directly or indirectly, whereby the modification may consist of (i) a radioactive marking, (ii) colored groups, (iii) fluorescent groups, (iv) chemoluminescent groups, (v) groups for immobilization at a solid phase and/or (vi) groups which permit an indirect or direct detection reaction using antibodies, antigens, enzymes and/or substances with an affinity to enzymes or enzyme complexes, or a combination of modifications according to two or more of the modifications listed under (i) to (vi).

32. Kit for the detection of microorganisms of the species Yersinia pestis/Yersinia pseudotuberculosis and for differentiation between Yersinia pestis and Yersinia pseudotuberculosis containing two or more nucleic acids according to claim 21.

33. The kit according to claim 32 containing in addition an amplification control for the amplification reaction.


SEQUENCE LISTING (13 sequences)

SEQ ID NO: 1 - DNA, 20 bp, Yersinia sp.
atcttactttccgtgagaag

SEQ ID NO: 2 - DNA, 20 bp, Yersinia sp.
cttggatgttgagcttccta

SEQ ID NO: 3 - DNA, 24 bp, Yersinia sp.
ggattattggttagatacggttac

SEQ ID NO: 4 - DNA, 22 bp, Yersinia sp.
ggtgatcccatgtacttaacat

SEQ ID NO: 5 - DNA, 16 bp, Yersinia sp.
ggcagagatgctaaag

SEQ ID NO: 6 - DNA, 20 bp, Yersinia sp.
atttggcagagatgctaaag

SEQ ID NO: 7 - DNA, 26 bp, Yersinia sp.
ctcccatggtgtgacgggcggtgtgt

SEQ ID NO: 8 - DNA, 17 bp, Yersinia sp.
tgtgacgggcggtgtgt

SEQ ID NO: 9 - DNA, 22 bp, Yersinia sp.
ctacttcttttgcaacccactc

SEQ ID NO: 10 - DNA, 21 bp, Yersinia sp.
agtcatcatggcccttacgag

SEQ ID NO: 11 - DNA, 20 bp, Yersinia sp.
gatgacgtcgtcttggctac

SEQ ID NO: 12 - DNA, 20 bp, Yersinia sp.
ggtctgcaatatcgcttctg

SEQ ID NO: 13 - DNA, 20 bp, Artificial
  ST probe
gactacggaattccgctgtc